SEQUENCE LISTING (32 sequences)

SEQ ID NO: 1 - RNA, 21 nt, Homo sapiens
cgucuaggccccccgaaccac

SEQ ID NO: 2 - RNA, 21 nt, Homo sapiens
gcagauccggggggcuuggug

SEQ ID NO: 3 - RNA, 21 nt, Homo sapiens
cucguccccuccggccguacc

SEQ ID NO: 4 - RNA, 21 nt, Homo sapiens
gagcaggggaggccggcaugg

SEQ ID NO: 5 - RNA, 21 nt, Homo sapiens
gggggggaggcaccucauuuu

SEQ ID NO: 6 - RNA, 21 nt, Homo sapiens
cccccccuccguggaguaaaa

SEQ ID NO: 7 - RNA, 21 nt, Homo sapiens
ggagaugaaggcgaaggcguc

SEQ ID NO: 8 - RNA, 21 nt, Homo sapiens
ccucuacuuccgcuuccgcag

SEQ ID NO: 9 - RNA, 21 nt, Homo sapiens
gacacugagacaccaauugac

SEQ ID NO: 10 - RNA, 21 nt, Homo sapiens
cugugacucugugguuaacug

SEQ ID NO: 11 - RNA, 21 nt, Homo sapiens
gggcagaacugcggcuaucgc

SEQ ID NO: 12 - RNA, 21 nt, Homo sapiens
cccgucuugacgccgauagcg

SEQ ID NO: 13 - DNA, 32 bp, Homo sapiens
cctgtggagaacctagggacaccatgagatcc

SEQ ID NO: 14 - DNA, 30 bp, Homo sapiens
cctaatatcaggatcggggtgagaacaatt

SEQ ID NO: 15 - DNA, 49 bp, Homo sapiens
cctcaaagaaaaaccaaacgtaacaccaacgggcgcgccatgattgaac

SEQ ID NO: 16 - DNA, 37 bp, Homo sapiens
cgatgctcgtatgcggaaacgaccttgtcgttatctg

SEQ ID NO: 17 - DNA, 7989 bp, Homo sapiens
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaagggcgcgccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtttaaacagaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaaccggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggaaggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagt

SEQ ID NO: 18 - DNA, 23 bp, Homo sapiens
  misc_feature: First strand of double stranded RNA. Second strand is SEQ ID NO 19.
cgucuaggccccccgaaccactt

SEQ ID NO: 19 - DNA, 23 bp, Homo sapiens
  misc_feature: Second strand of double stranded RNA. First strand is SEQ ID NO 18. This sequence was entered in reverse order because it starts with 3′.
gugguucggggggccuagacgtt

SEQ ID NO: 20 - DNA, 23 bp, Homo sapiens
  misc_feature: First strand of a double stranded RNA. Second strand is SEQ ID NO 21.
cucguccccuccggccguacctt

SEQ ID NO: 21 - DNA, 23 bp, Homo sapiens
  misc_feature: Second strand of double stranded RNA. First strand is SEQ ID NO 20. This strand is entered in reverse order because it begins with 3′.
gguacggccggaggggacgagtt

SEQ ID NO: 22 - DNA, 23 bp, Homo sapiens
  misc_feature: This is the first strand of a double stranded RNA. The second strand is SEQ ID NO 23.
gggggggaggcaccucauuuutt

SEQ ID NO: 23 - DNA, 23 bp, Homo sapiens
  misc_feature: Second strand of double stranded RNA. First strand is SEQ ID NO 22. This strand is entered in reverse order because it begins with 3′.
aaaaugaggugccuccccccctt

SEQ ID NO: 24 - DNA, 23 bp, Homo sapiens
  misc_feature: First strand of double stranded RNA. Second strand is SEQ ID NO 25.
ggagaugaaggcgaaggcguctt

SEQ ID NO: 25 - DNA, 23 bp, Homo sapiens
  misc_feature: Second strand of double stranded RNA. First strand is SEQ ID NO 24. This strand is entered in reverse order because it begins with 3′.
gacgccuucgccuucaucucctt

SEQ ID NO: 26 - DNA, 23 bp, Homo sapiens
  misc_feature: First strand of double stranded RNA. Second strand is SEQ ID NO 27.
gacacugagacaccaauugactt

SEQ ID NO: 27 - DNA, 23 bp, Homo sapiens
  misc_feature: Second strand of double stranded RNA. First strand is SEQ ID NO 26. This strand is entered in reverse order because it begins with 3′.
gucaauuggugucucaguguctt

SEQ ID NO: 28 - DNA, 23 bp, Homo sapiens
  misc_feature: First strand of double stranded RNA. Second strand is SEQ ID NO 29.
gggcagaacugcggcuaucgctt

SEQ ID NO: 29 - DNA, 23 bp, Homo sapiens
  misc_feature: Second strand of double stranded RNA. First strand is SEQ ID NO 28. This strand is entered in reverse order because it begins with 3′.
gcgauagccgcaguucugccctt

SEQ ID NO: 30 - DNA, 23 bp, Homo sapiens
ggacaucuagcgguagcccactt

SEQ ID NO: 31 - DNA, 23 bp, Homo sapiens
gagagucagucagcuaaucactt

SEQ ID NO: 32 - DNA, 23 bp, Homo sapiens
aacaagucucguauguaguggtt

(19) United States
(12) Patent Application Publication
(10) Pub. No.: US 2005/0043266 A1
(43) Pub. Date: Feb. 24, 2005

(54) Short interfering RNA as an antiviral agent for hepatitis C

(76) Inventors: Sumedha Jayasena, Thousand Oaks, CA (US); Christopher Donald Richardson, Toronto (CA)

Correspondence Address: U.S. Patent Operations/KLN;Dept. 4300, M/S 27-4-A, AMGEN INC., One Amgen Center Drive, Thousand Oaks, CA, 91320-1799, US

(21) Appl. No.: 10/897,648

(22) Filed: Jul. 22, 2004

Related U.S. Application Data
(60) Provisional application No. 60/490,204, filed on Jul. 25, 2003.

(51) Int. Cl. A61K 48/00; C07H 21/02

(52) U.S. Cl. 514/44; 536/23.1

(57) ABSTRACT

Hepatitis C virus (HCV) is a major cause of chronic liver disease and affects over 270 million individuals worldwide. The HCV genome is a single-stranded RNA that functions as both a messenger RNA and replication template, making it an attractive target for the study of RNA interference. Double-stranded short interfering RNA (siRNA) molecules designed to target the HCV genome are disclosed herein.

This application claims the benefit of U.S. Provisional Application No. 60/490,204 filed Jul. 25, 2003, which is hereby incorporated by reference.

### FIELD OF THE INVENTION

This invention is in the field of pharmaceutical agents and specifically relates to compounds, compositions, uses and methods for treating Hepatitis C Virus (HCV) and related disorders.

### BACKGROUND OF THE INVENTION

RNA interference is a phenomenon in which short, double-stranded RNA molecules induce sequence-specific degradation of homologous single-stranded RNA (1). In plants and insects, RNA interference activity plays a role in host cell protection from viruses and transposons (2, 3). From a practical perspective, RNA interference is proving to be a very powerful technique to “knock-down” specific genes in order to evaluate their physiological roles Caenorhabditis elegans (1, 4), Drosophila melanogaster (5), and humans (6).

In plants and invertebrates, RNA interference can be induced through transfection or microinjection of long double-stranded RNA (1, 7). The dsRNA is cleaved into short 19 to 23 nt RNA fragments known as short interfering RNAs (siRNA) (8). Short interfering RNAs are incorporated into a ribonuclease enzyme complex known as the RNA Induced Silencing Complex (RISC). The antisense strand of siRNA within the RISC and serves as a guide for sequence-specific degradation of homologous messenger RNAs. Only small RNA molecules, <30 bases in length, can be used to exclusively induce RNA interference in mammalian cells, since larger molecules also activate the non-specific dsRNA-dependent response (9, 10). In plants and nematodes, RNA interference activity is long-term and disseminates throughout the organism via an uncharacterized amplification mechanism. In mammalian cells, amplification activity appears to be absent, and interference activity is transient, lasting for only 3 to 5 days. More recently, DNA expression vectors have been developed to express hairpin or duplex small interfering RNAs. These vectors employ the type III class of RNA polymerase promoters in order to drive the expression of siRNA molecules (11-14). In addition, stable cell lines containing siRNA expression plasmids have been produced in order to induce RNA interference over longer durations (13, 15).

The potential of using RNA interference activity for treatment of viral diseases and cancer has aroused a great deal of interest in the scientific community. Other laboratories have reported the use of RNA interference activity in cultured cells infected with HIV, HPV, polio, or containing a variety of cancer genes (16-21). Hepatitis C is a major health concern and an estimated 3% of the world's population (270 million individuals) is chronically infected with this viral pathogen. It is estimated that 40-60% of infected individuals progress to chronic liver disease and many of these patients ultimately require liver transplantion (22). Currently, the only treatment available for patients with chronic hepatitis C infections consists of combination therapy with interferon and ribavirin. The standard therapy has a poor response rate (23) and thus there is a great need for the development of new treatments for hepatitis C virus infections. We have investigated the effect of RNA interference activity on the replication of the hepatitis C virus (HCV) using the recently established replicon system (24-26). We have identified two siRNAs capable of dramatically reducing viral protein and RNA synthesis. In addition, we also showed that RNA interference could protect naive Huh-7 cells from challenge with the replicon RNA. Finally, the duration of protective interference activity was extended beyond 3 weeks by expressing siRNAs from a bicistronic expression vector that replicated as an episome.

RNA interference represents an exciting new technology that could have applications in the treatment of viral diseases. Previous reports have shown that siRNAs directed against the HIV genome can effectively inhibit virus production in model cell culture systems (1, 19, 20, 32). In addition, RNA interference activity directed towards the major HIV receptor protein, CD4, led to decreased entry of HIV into cells (19). However, replication of HIV occurs through an integrated DNA genome, representing a situation where RNA interference is ineffective in clearing the virus. On the other hand, the HCV genome is a (+) sense single-stranded RNA that functions as both the viral messenger RNA and a template for RNA replication via a negative-strand intermediate (33). This situation suggests HCV could be a particularly attractive target for RNA interference therapy that could eliminate viral RNA from the infected cell and potentially cure a patient of hepatitis.

### SUMMARY OF THE INVENTION

The present invention provides isolated double-stranded RNA sequences that are effective as antiviral agents for hepatitis C.

The present invention further provides isolated dsRNA molecules useful in the treatment of HCV.

### DESCRIPTION OF THE FIGURES

FIG. 1A shows a schematic diagram of the HCVrepAB12neo replicon RNAs showing the approximate locations of the siRNA target sequences.

FIG. 1B shows a Western blot analysis of HCV non-structural protein levels in AB12-A2 cells 72 h after electroporation.

FIG. 1C shows a Northern blot analysis of negative strand HCV replicon RNA levels in siRNA treated replicon cells 48 h after induction of RNA interference.

FIG. 1D shows a Northern blot analysis of HCV replicon RNA levels in AB12-A2 cells 48 h after electroporation of siRNAs.

FIG. 2A shows Huh-7 cells that were electroporated with 10 ng HCVrepAB12neo alone (HCVrepAB12neo), or with 10 ng HCVrepAB12neo in a solution containing 100 nM of the indicated siRNA molecules (HCVrepAB12neo+siRNA).

FIG. 2B shows a histogram reflecting the average number of colonies that grew after repeating the experiment. Error bars show the standard deviations of the average of three independent experiments.

FIG. 2C shows a histogram reflecting the effect of siRNA on transient luciferase expression from an HCV replicon carrying the luciferase reporter gene.

FIG. 3A shows Huh-7 cell that were electroporated with 100 nM of either siRNA 6367 or negative control siRNA 6367 mm. The top row shows a time course of colony formation in cells electroporated with the negative control siRNA 6367 mm and the bottom row shows a time course of colony formation by cells electroporated with HCV specific siRNA 6367

FIG. 3B shows a histogram showing the relative percent of colony formation when HCV replicon RNA was electroporated 24, 72, 96 and 120 h after induction of RNA interference.

FIG. 4A shows a schematic diagram of pCEP4d plasmid that replicates in the cell as a multi-copy episome and contains dual H1 promoters to drive expression of the complementary siRNA strands.

FIG. 4B shows a Western blot evaluation of the levels of HCV nonstructural protein NS3 in AB12-A2 cells.

FIG. 4C shows Huh-7 cells were transfected with pCEP4d empty vector (top), pCEP4d6367 (middle) or with pCEP4d6367 mm (bottom) and grown for 21 days in media containing hygromycin.

FIG. 5 shows Table 1 describing the siRNAs used in this study.

FIG. 6 shows the HCV replicon RNA, I377/NS3-3′UTR. (7898bp RNA)(Accession No. AJ242652)used herein (SEQ ID NO: 17).

### DETAILED DESCRIPTION OF THE INVENTION

We have demonstrated that HCV replicon RNA is susceptible to RNA interference in a human hepatoma cell line (Huh-7). Introduction of two different siRNAs into target cells that contained HCV replicon RNA caused a dramatic decrease in the levels of viral proteins and RNA. This effect was likely due to the degradation of HCV messenger RNA by the RISC endonuclease. HCV specific RNA interference activity also led to a reduction in the levels of HCV (−) strand replication intermediate RNA and allows for the possibility that replicating HCV RNA may also be susceptible to degradation by RISC. We do not know the effect of RNA interference on HCV immediately after virus entry into cells since an efficient cell culture system for growth of HCV is not available at this time. However, we have shown that up to a 99% reduction in the efficiency of HCV replicon colony formation when interference activity was induced concurrent with replicon RNA entry into cells. Thus RNA interference protects cells from “infection” by HCV replicon RNA. Since the early events of an HCV infection include translation of the newly uncoated genomic RNA, it is likely that the viral RNA will also be susceptible to RNA interference at this time. However, this remains to be determined.

The efficacy of each of the six short interfering RNAs that were designed to target different regions of the HCV replicon RNA varied greatly. This is in agreement with siRNAs targeted to other genes (19). The reasons that certain siRNAs did not induce HCV specific RNA interference are not known, but one could speculate several possibilities. SiRNAs that are inefficient in RNAi response may target regions of RNA that are inaccessible to RISC due to either secondary structure or protein binding or both. Alternatively, these siRNAs may not form RISCs that are productive in eliciting RNA interference.

Due to the great variability in RNA sequences between different quasi-species and genotypes of HCV, for therapeutic applications it may be necessary to include several different combinations of siRNA in order to target a particular region of the genome. In addition, the high mutation rate of HCV that is apparent during replication makes the appearance of escape mutants from RNA interference a distinct possibility as was seen for poliovirus (16). However, the development of viral resistance to RNA interference may not merely be limited to the production of escape mutants through sequence divergence. Many plant viruses (2, 34) and at least one animal virus (35) synthesize gene products that appear to block RNA interference activity. Whether HCV possesses such an activity remains to be determined (35).

The utility of siRNA as a therapy against HCV infection will depend on the development of efficient delivery systems that induce long lasting RNA interference activity. HCV is an attractive target for its localization in the liver, an organ that can be readily targeted by nucleic acids molecules and viral vectors. In the future, chemically modified synthetic siRNAs with improved resistance to nucleases coupled with enhanced duration of RNA interference may become a possibility for therapeutic applications. On the other hand, gene therapy offers another possibility to express siRNAs that target HCV in patient's liver. For the first time, our laboratory has produced cells that exhibit stable RNA interference directed against a virus. We constructed a self-contained episomal expression vector that contains the oriP origin of replication, a coding sequence for EBNA1 protein that is required for episome maintenance, and two H1 tandem promoters that drive the synthesis of each of the siRNA strands. This expression vector extended expression of the siRNA from 72 h to over 3 weeks. Others laboratories have observed long acting RNA interference through the establishment of stable cell lines that constitutively express specific siRNAs (13, 15, 36). Two recent reports have described the use of recombinant adenoviruses and retroviruses to deliver and express siRNA in culture. The adenovirus was also used to deliver siRNAs to the livers of mice (37, 38). Similar vectors could eventually be used from a prophylactic or therapeutic standpoint to evaluate the effects of siRNA on HCV replication in model systems such as chimpanzees and mice with chimeric human livers (39). Based upon the experiments disclosed herein, the use of siRNA as a treatment for HCV infections, has great potential for use alone, or in combination with conventional interferon/ribavirin therapy as a means to decrease virus loads and eventually clear the persistent virus from its host.

### EXAMPLES

### Example 1

Preparation of Cell Culture

The cell line Huh-7 (27) was kindly provided by Dr. Stanley M. Lemon (The University of Texas Medical Branch at Galveston, Galveston, Tex.) and were routinely grown in Dulbecco's minimal essential media supplemented with nonessential amino acids, 100 U/mL of penicillin, 100 μg/mL of streptomycin, and 10% fetal calf serum (FCS, Wisent Inc, Montreal, Canada). Cell lines carrying HCV replicons were grown in medium containing 800 μg/ml of G418 active ingredient (Geneticin: Gibco/Invitrogen, Carlsbad, Calif.).

Construction of HCV Replicons and pCEP4-H1/H1 Expression Vector and Synthesis of siRNAs

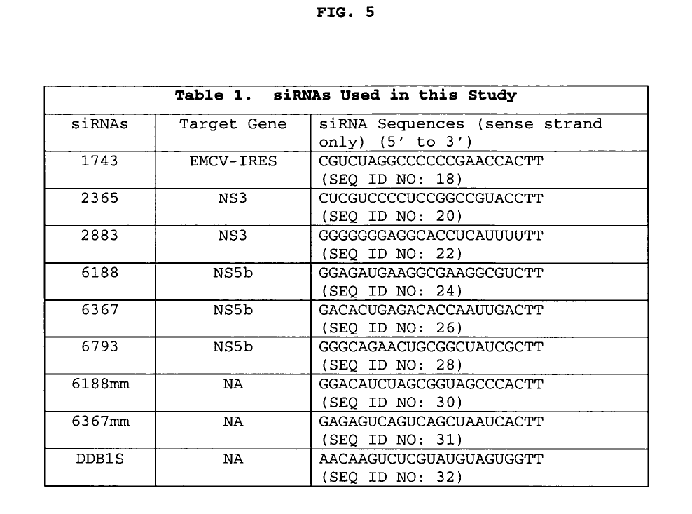

Plasmids pHCVrep1b BB7 (25) and p90/HCV FL-long pU (28) were provided by Dr. Charles M Rice (Center for the Study of Hepatitis C, The Rockefeller University, New York, N.Y.). The plasmid pHCVrepAB12 was made by adding two additional adaptive mutations, E1202G and T1280I (26), to the NS3 coding region, and an additional 12 nucleotides of the HCV IRES (29). Sequence changes were made using The Quickchange Mutagenesis Kit (Stratagene, La Jolla, Calif.). One strand of each complementary pairs of mutagenic primer is shown. Adaptive mutations E1202G and T1280I were introduced through mutagenesis of nucleotides A2330G and C2564T of the replicon sequence using primers CCTGTGGAGAACCTAGGGACACCATGAGATCC (SEQ ID NO: 13) and CCTAATATCAGGATCGGGGTGAGAACAATT (SEQ ID NO: 14). The 12 nucleotide insert was added using primer CCTCAAAGAAAAACCAAACGTAACACCAACGGGCGCGCCATGATTGAAC (SEQ ID NO: 15). The negative control replicon pHCVrepAB12mut contain a GDD-GND mutation in the NS5b polymerase coding sequence that was made using the primer CGATGCTCGTATGCGGAAACGACCTTGTCGTTATCTG (SEQ ID NO: 16). pHCVrepAB12Luc, was made by removing the neomycin gene from pHCVrepAB12 by digestion with AscI and PmeI, and inserting the luciferase gene, which had been amplified from the plasmid pGL2 (Promega, Madison, Wis., USA) using standard techniques. The plasmid pCEP4d was made by digesting pCEP4 (InVitrogen, Carlsbad, Calif.) with PvuII and SnaB1 and religating to remove the CMV IE promoter element. A DNA insert encoding tandem H1 promoters driving the sense and antisense siRNAs 6367 and 6367 mm was made by PCR. A detailed description of the cloning method is available on request. All plasmid constructs were sequenced for confirmation. Synthetic siRNA duplexes described in Table 1 (FIG. 5) were obtained from Dharmacon. SiRNAs 6188 mm and 6367 mm are negative control duplexes each containing 6 nucleotide mismatches in the target sequence.

Production of Monoclonal Antibodies Against HCV 1a H77 NS4a/3 and NS5b

The plasmids pETNS4A/NS3 and pETNS5B, containing the coding sequences for NS4A/NS3 and NS5B, were transformed into BL21 bacteria. The His-tagged proteins were purified (Amersham Pharmacia, Piscataway, N.J.) and injected into mice. Hybridoma cell lines were produced and screened using standard methods (30).

In Vitro Transcription

HCV replicon RNAs were transcribed in vitro using the T7-Megascript in-vitro transcription kit (Ambion, Austin, Tex.) according to the instructions of the manufacturer. After RNA synthesis, the DNA template was removed by 3 repeated digests with 0.2 U/μL of DNAse I enzyme at 37° C. for 30 min.

Electroporation of HCV Replicon and siRNA, and Selection with G418

Cells were electroporated using the protocol described by Lohmann et al (24). Either 10 ng of HCVrepAB12neo/HCVrepAB12neomut replicon RNA or 10 μg of HCVrepAB12Luc/HCVrepAB12Lucmut RNA were electroporated into naïve Huh-7 cells, alone or with 100 nM siRNA. AB12-A2 cells were electroporated with 1 μM siRNA. Plasmid pcDNA1uc (1 μg) was added to each sample to determine electroporation efficiency. If the cells were to be assayed for colony formation, they were transferred to 8 mL DMEM and seeded into one 10 cm diameter tissue culture dish. 24 h later and every 3 to 4 days subsequently, the media was replaced with fresh DMEM supplemented with 800 μg/mL G418 until colonies were visible. Colonies were fixed stained with 0.1% gentian violet. To screen for luciferase expression three 35 mm plates were seeded, each with 5% of the electroporated cells. At 3, 48 and 72 h post-electroporation, the cells were harvested and assayed for luciferease activity (Promega, Madison, Wis.). The luciferase levels at 3 h post-electroporation were used to correct for transfection efficiency.

Transfection of Plasmid DNA into Huh-7 Cells

Huh-7 cells were transfected with pCEP4d plasmids expressing siRNA 6367, siRNA 6367 mm or with no insert. The plasmids were transfected into Huh-7 cells using Lipofectamine2000 (Invitrogen, Carlsbad, Calif.) and suggested method. Medium containing 75 μg/mL hygromycin (Invitrogen, Carlsbad, Calif.) was added to the cells 24 h post-transfection.

RNA Purification and Northern Blot Analysis

RNA samples were purified from Huh-7 cells using Trizol reagent (Life Technologies, Invitrogen, Carlsbad, Calif.). Total RNA (5 μg) was treated with glyoxal and subjected to electrophoresis in a 0.9% agarose gel using standard techniques (31). The gels were transferred to Hybond N+ nylon membrane (Amersham Pharmacia, Piscataway, N.J.) and probed with ³²P labeled neomycin resistance gene DNA which had been labeled using the Ready-To-Go DNA labeling kit (Amersham Pharmacia, Piscataway, N.J.). An HCV sense strand specific riboprobe was made using Hind III linearized pHCVrepAB12 replicon plasmid as a template for use in the Riboprobe T7 system with α³²P UTP (Promega, Madison, Wis.).

SDS-PAGE and Western Blot Analysis

Equal numbers of naive or replicon-containing Huh-7 cells were lysed in SDS sample buffer 72 h after electroporation with siRNAs. Protein was electophoresed on a Polyacrylamide gel (Novex Invitrogen, Carlsbad Calif.) and transferred to Hybond-C Extra supported nitrocellulose membrane (Amersham Pharmacia, Piscataway, N.J.). The blots were probed with monoclonal anti-NS3, anti-NS5b and anti-actin using standard methods. Proteins were visualized using enhanced chemiluminescence (ECL, Amersham Pharmacia, Piscataway, N.J.).

### Example 2

Construction of HCV Replicon used in the Study

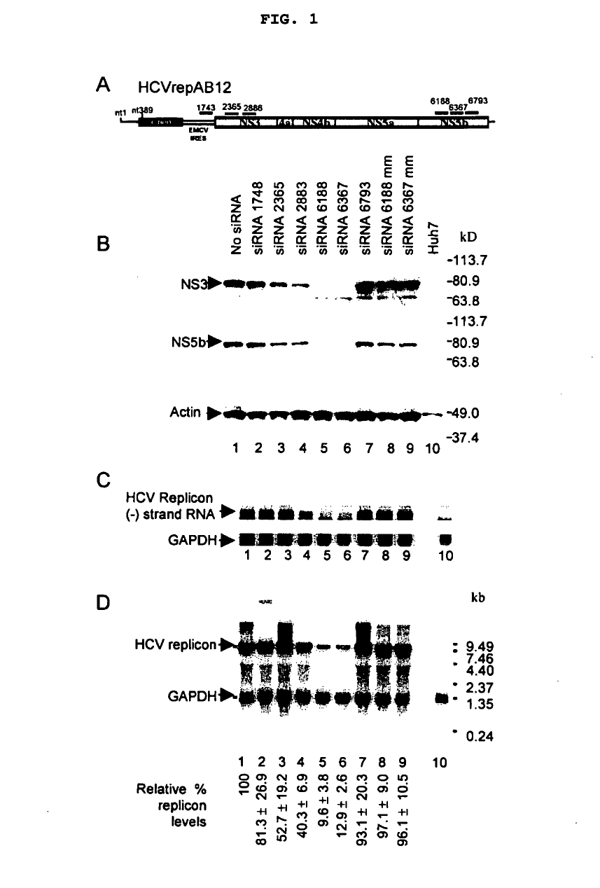

The design of the bicistronic HCV replicon used in this study is shown in FIG. 1A. The HCVrepBB7 replicon construct was obtained from Dr. Charles Rice and contained the adaptive mutation S2204I (25). We constructed an enhanced replicon construct by introducing two additional adaptive mutations, E1202G and T1280I (26) and extending the HCV IRES by 12 nucleotides (29). The enhanced replicon construct, HCVrepAB12 had a colony forming efficiency of 1×105 colonies per μg of RNA, which is a 1700-fold improvement over the efficiency of HCVrepBB7 in our hands. A G418 resistant cell clone, AB12-A2, was isolated, amplified and screened for the presence of the replicon RNA and absence of replicon DNA by PCR.

### Example 3

RNA Interference Silences HCV Subgenomic Replication and Gene Expression

Six siRNAs were designed to trigger RNA interference through homology to specific regions of the HCV subgenomic replicon (FIG. 1A, FIG. 5). These six siRNA triggers are:

| | |
|---|---|
| 1743 | |
| 5′- CGU CUA GGC CCC CCG AAC CAC - dTdT - 3′ | (SEQ ID NO 18) |
| 3′- dTdT - GCA GAU CCG GGG GGC UUG GUG - 5′ | (SEQ ID NO 19) |
| 2365 | |
| 5′- CUC GUC CCC UCC GGC CGU ACC - dTdT - 3′ | (SEQ ID NO 20) |
| 3′- dTdT - GAG CAG GGG AGG CCG GCA UGG - 5′ | (SEQ ID NO 21) |
| 2883 | |
| 5′- GGG GGG GAG GCA CCU CAU UUU - dTdT - 3′ | (SEQ ID NO 22) |
| 3′- dTdT - CCC CCC CUC CGU GGA GUA AAA - 5′ | (SEQ ID NO 23) |
| 6188 | |
| 5′- GGA GAU GAA GGC GAA GGC GUC - dTdT - 3′ | (SEQ ID NO 24) |
| 3′- dTdT- CCU CUA CUU CCG CUU CCG CAG - 5′ | (SEQ ID NO 25) |
| 6367 | |
| 5′- GAC ACU GAG ACA CCA AUU GAC - dTdT - 3′ | (SEQ ID NO 26) |
| 3′- dTdT- CUG UGA CUC UGU GGU UAA CUG - 5′ | (SEQ ID NO 27) |
| 6793 | |
| 5′- GGG CAG AAC UGC GGC UAU CGC - dTdT - 3′ | (SEQ ID NO 28) |
| 3′- dTdT- CCC GUC UUG ACG CCG AUA GCG - 5′ | (SEQ ID NO 29) |

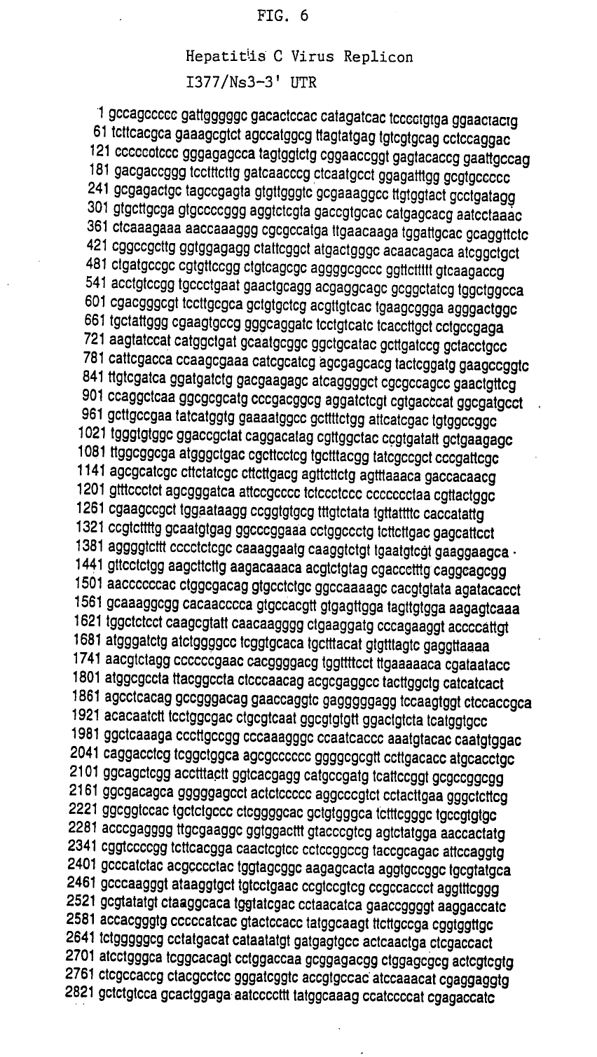

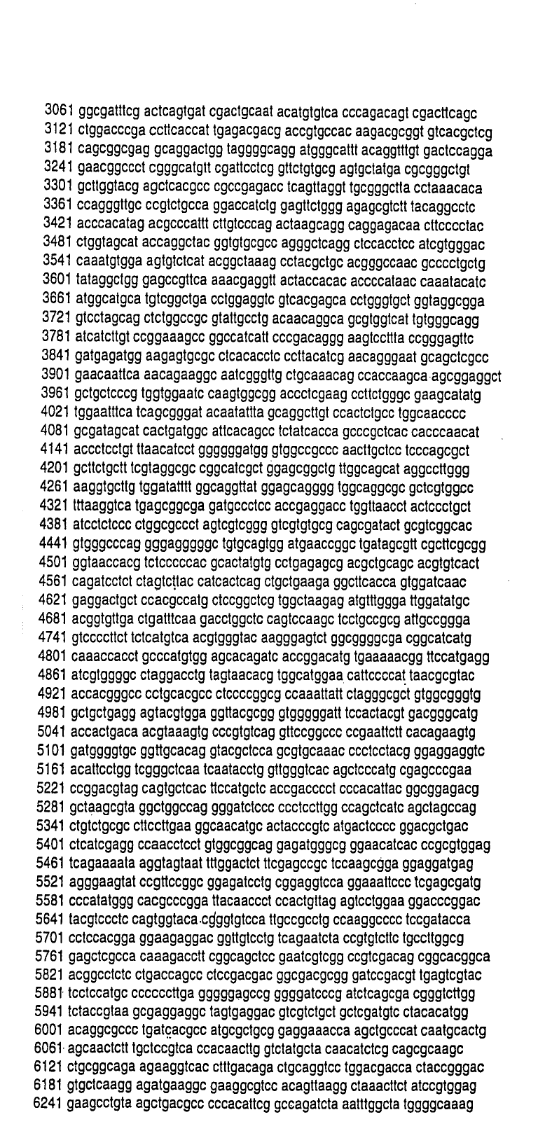

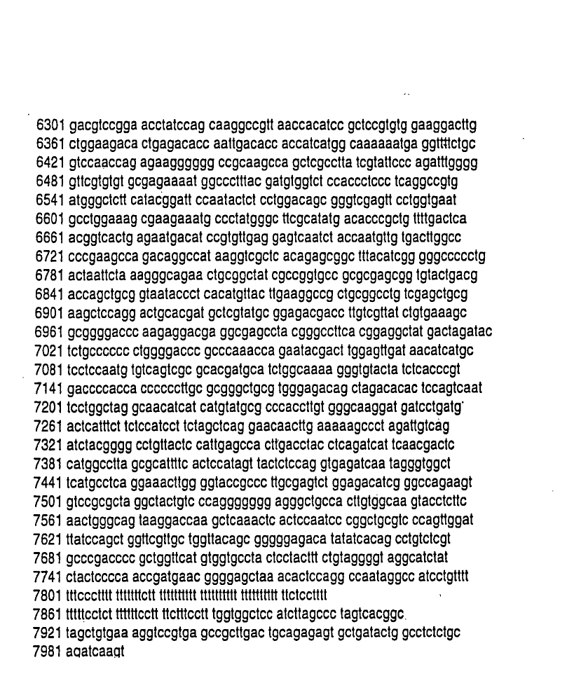

As used herein, each siRNA trigger carried two dT residues at their 3′ ends and was produced by chemical RNA synthesis. The designated number (in bold) in each case reflects the position within the HCV replicon RNA, I377/NS3-3′ UTR. (7898bp RNA) (Accession No. AJ242652) (FIG. 6).

The effect of RNA interference on HCV protein and RNA levels was examined by western and northern blot analysis of samples of the HCV replicon cell line (AB12-A2). Of the six triggers tested, siRNAs 6188 and 6367 elicited the most potent effect. At 72 h post-electroporation, HCV non-structural proteins NS3 and NS5b levels were below the detection limit by western blot analysis (FIG. 1B, Lanes 5 and 6) and the levels of replicon RNA were 9.6 and 12.9%, respectively, when compared with the levels of replicon RNA in control cells electroporated in the absence of siRNA (FIG. 1D, Lanes 5 and 6).

FIG. 1B shows a Western blot analysis of HCV non-structural protein levels in AB12-A2 cells 72 h after electroporation. Samples were electroporated in the absence of siRNA (Lane 1, no siRNA), with one of 6 HCV sequence-specific siRNAs (Lanes 2 to 6), or with a control siRNA containing mismatched nucleotides (Lanes 8 and 9, siRNA 6188 mm and siRNA 6367 mm). A sample from the parental Huh-7 cells are shown (Lane 10). Blots were probed with monoclonal antibodies to either NS3 (top) or NS5b (middle). A third blot was probed with anti-actin (bottom) to control for protein loading. Protein size markers are shown on the right side of the figure.

FIG. 1C shows a Northern blot analysis of negative strand HCV replicon RNA levels in siRNA treated replicon cells 48 h after induction of RNA interference. RNA was purified from a portion of the samples described in B. The northern blot was probed with 32P labeled (−) strand-specific riboprobe to detect the negative strand of HCV replicon RNA and a 32P labeled GAPDH DNA to control for RNA loading. The locations of the HCV replicon (−) strand RNA and control GAPDH RNA are indicated.

FIG. 1D shows a Northern blot analysis of HCV replicon RNA levels in AB12-A2 cells 48 h after electroporation of siRNAs. The samples analyzed by northern blot are identical to those described in B. RNA size markers are shown on the right side of the figure and the RNA bands corresponding to HCV replicon RNA and GAPDH mRNA are indicated on the left. HCV replicon and GAPDH RNA levels on northern blots were quantitated by phosphorimage analysis. The average level of HCV RNA present in each sample is given below each lane as a percentage, relative to the levels seen in cells not treated with siRNA. Percent standard deviations are given based on the average of three independent experiments.

Electroporation of negative control siRNAs containing 6 mismatched nucleotides (FIG. 1D, Lane 1) did not have any effect on the levels of HCV replicon RNA or HCV non-structural proteins in AB12-A2 cells. These results indicate that the effects of the siRNAs on HCV replicon protein and RNA levels were sequence-specific and not caused by induction of nonspecific host defense pathways (FIG. 1D, Lanes 1, 7 and 8). Thus the effects of siRNAs on HCV protein and RNA levels appear to be the result of siRNA directed degradation of the HCV replicon RNA by RISC. The other four siRNAs used in this experiment were less effective or had no significant effect (FIG. 1B, Lanes 2, 3, 4, 7). The relative effectiveness of the siRNA trigger sequences was confirmed by quantification of HCV replicon RNA levels by real-time reverse-transcription PCR. Using this method the relative reduction in HCV replicon RNA levels was more dramatic, with a 99% and 94% decrease in replicon RNA levels, following treatment with siRNA 6188 and siRNA 6367 (data not shown). RNA interference also reduced the levels of negative strand HCV replicon RNA (FIG. 1C) indicating that RISC endonuclease activity may also target and degrade the (−) strand of the HCV replication intermediate.

### Example 4

HCV-Specific siRNA Protects Cells from Challenge with the HCV Subgenomic Replicon

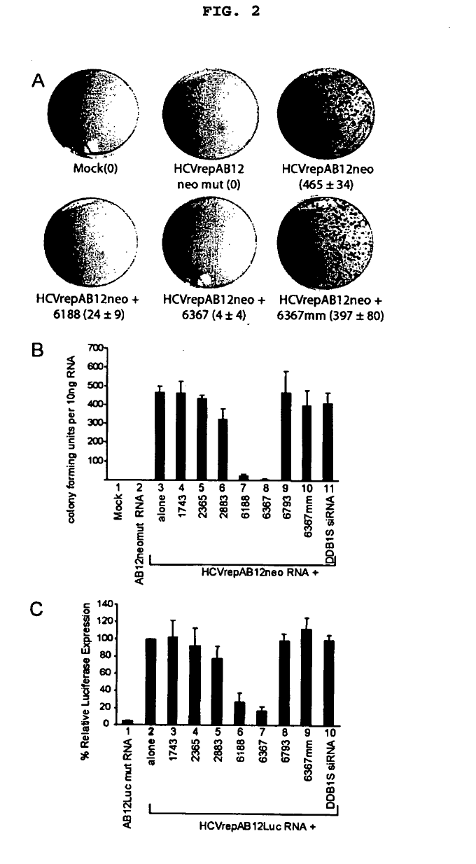

Electroporation of 10 ng of HCVrepAB12neo replicon RNA into Huh-7 cells resulted in the growth of about 465 G418 resistant colonies (FIG. 2A, B). FIG. 2A shows Huh-7 cells that were electroporated with 10 ng HCVrepAB12neo alone (HCVrepAB12neo), or with 10 ng HCVrepAB12neo in a solution containing 100 nM of the indicated siRNA molecules (HCVrepAB12neo+siRNA). Negative control samples were electroporated with 10 ng non-replicating replicon RNA (HCVrepAB12neomut), or in the absence of RNA (mock). Cells were selected in G418 until colonies were visible. The colonies were enumerated after staining. FIG. 2B shows the experiment described above repeated 3 times and the average number of colonies that grew in each sample was plotted as a histogram. Error bars show the standard deviations of the average of three independent experiments.

Triggering HCV-specific gene silencing by co-electroporation of siRNAs with replicon RNA caused a dramatic decrease in the number G418 resistant colonies when siRNAs 6188 and 6367 were used (FIGS. 2A, B). The efficacy of individual siRNAs mirrored the results seen in the previous experiments. Small interfering RNAs 6188 and 6367 were the most potent inhibitors and caused a 95% and 99% reduction, respectively, in the numbers of G418 resistant colonies formed. The siRNAs 1748, 2365, 2883, and 6793 had only marginal effects on colony formation (FIGS. 2A and B). Control siRNAs, siRNA 6367 mm and DDB1S (a nonspecific siRNA) gave no significant reduction in G418 resistant colony formation. A control replicon in which the NS5B gene was mutated and rendered nonfunctional (HCVrepAB12neo-mut) could not produce G418 resistant colonies following electroporation into Huh-7 cells.

Similar results were seen in a transient assay designed to measure the stability and replication of the HCV subgenomic replicon through the use of a luciferase assay (FIG. 2C). A luciferase reporter gene was inserted into the replicon RNA in place of the neomycin resistance gene to produce HCVrepAB12Luc. Stability of the replicon was determined by measuring luciferase expression levels at 72 h post electroporation of the various siRNAs. Again, the effects of the siRNA triggers on luciferase expression levels were similar to those seen in the previous assays (FIG. 2C).

FIG. 2C shows the effect of siRNA on transient luciferase expression from an HCV replicon carrying the luciferase reporter gene. Huh-7 cells were electroporated with 10 μg of control nonreplicating luciferase replicon RNA (HCVrepAB12Lucmut), replicating replicon RNA (HCVrepAB12Luc) alone, or in a solution containing the indicated siRNA. Luciferase levels were measured at 3 and 72 h post-electroporation. The levels at 3 h post-infection were used to estimate electroporation efficiencies. The luciferase levels measured at 72 h from the HCVrepAB12Luc RNA alone were defined as 100% and the luciferase levels measured in the other samples are expressed as relative percentages. The data represents the average of three independent experiments and error bars represent the standard deviations.

Small interfering RNAs 6188 and 6367 were the most potent inhibitors and led to a reduction in relative luciferase expression levels to 27% and 16%, respectively, compared to controls in which siRNA was absent (FIG. 2C). Control siRNA 6367 mm and DDB1S, had no effects upon luciferase expression. These data suggest that co-electroporation of replicon RNA together with certain siRNAs can induce strong RNA interference activity. In the case of the most effective siRNA sequences HCV replicon colony growth was almost totally abolished.

### Example 5

Duration of RNA Interference Activity on HCV Subgenomic Replicon Triggered by Synthetic siRNAs

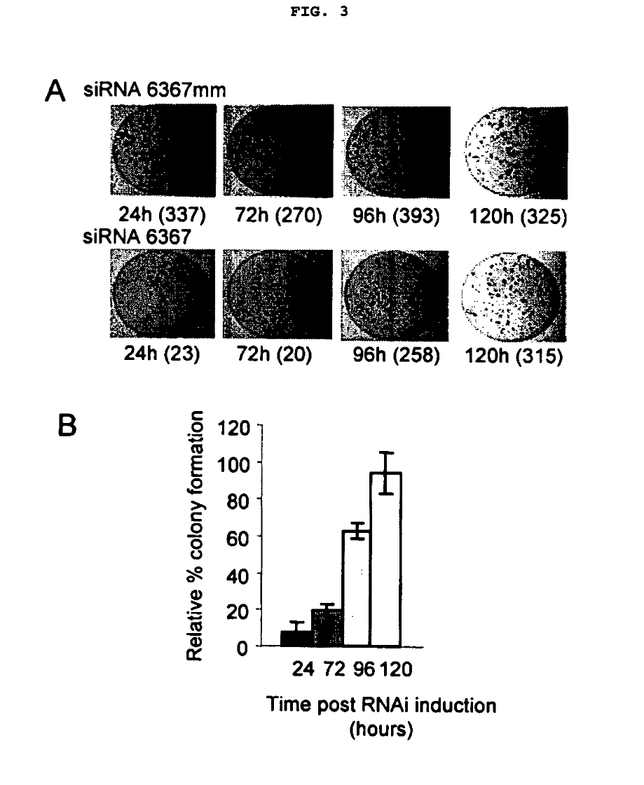

We investigated the duration of RNA interference activity on the HCV replicon in Huh-7 cells by first, introducing siRNA 6367 or control siRNA 6367 mm into Huh-7 cells by electroporation to induce RNA interference and then, at various times after electroporation, the cells were re-electroporated with 10 ng of HCVrepAB12 replicon RNA to assess the potency of interference activity at that particular time. Negative control cells were electroporated with siRNA 6367 mm (FIG. 3A, top row) and the numbers of colonies that formed (ranging between 270 to 393) did not change significantly over the duration of the experiment.

FIG. 3A shows Huh-7 cells that were electroporated with 100 nM of either siRNA 6367 or negative control siRNA 6367 mm. At 24, 72, 96 or 120 h following induction of interference, each population of cells was challenged with 10 ng of HCVrepAB12 replicon RNA, and then grown in media containing G418 until colonies were visible. The colonies that grew after a time course experiment are shown. The top row shows a time course of colony formation in cells electroporated with the negative control siRNA 6367 mm and the bottom row shows a time course of colony formation by cells electroporated with HCV specific siRNA 6367

The small variation in the colony numbers between each experiment reflected differences in electroporation efficiencies. However, in cells electroporated with siRNA 6367 RNA, the interference activity was strong at early times after induction and became weaker over time, as evidenced by the increase in numbers of G418 resistant colonies that grew when replicon RNA was electroporated 96 and 120 h after induction of interference (FIG. 3A, bottom row). The combined results of 3 replicate experiments are shown graphically (FIG. 3B). FIG. 3B reflects a histogram showing the relative percent of colony formation when HCV replicon RNA was electroporated 24, 72, 96 and 120 h after induction of RNA interference. The number of G418 resistant colonies that grew from cells that had been electroporated with control siRNA 6367 mm was defined as 100%, and the number of colonies that formed on plates with cells treated with siRNA 6367, are plotted relative to this value. Data represents the averages of 3 experiments for each time point. Error bars reflect the standard deviation from the average.

When HCV replicon RNAs were electroporated 24 or 72 h following induction of RNA interference with siRNA 6367, the effect of gene silencing on the HCV subgenomic replicon was potent, and caused a 92% and 80% reduction in the number of G418 resistant colonies (FIG. 3B, 24 and 72 h). However, at 96 h following introduction of siRNA 6367 to the cells, there was considerably less RNA interference activity and was insignificant by 120 h post induction (FIG. 3b, 96 and 120 h). As has been seen in other mammalian systems, the effect of RNA interference mediated by exogenously added synthetic siRNAs is short lived and appeared to extend to 96 h in our experiments.

### Example 6

Prolonged Duration of RNA Interference by Bicistronic Plasmids Expressing Complementary Short Interfering RNAs

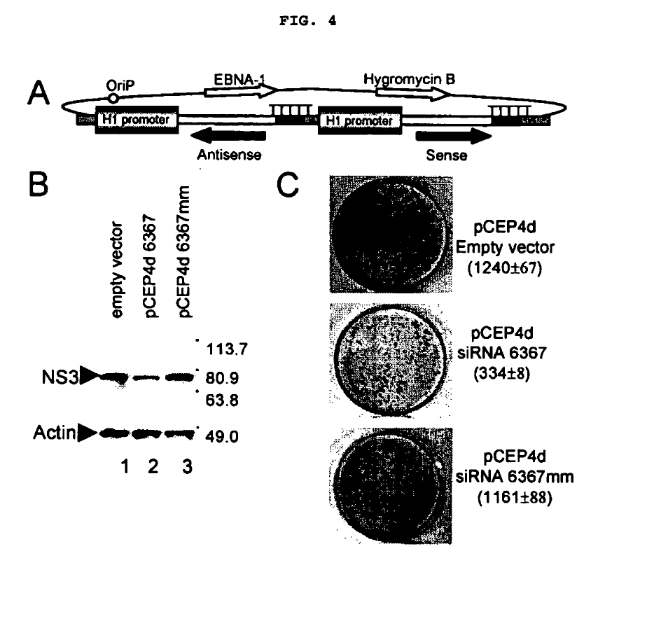

RNA interference was induced in Huh-7 liver cells by transfecting cells with a vector that expressed complementary strands of an siRNA under control of 2 separate H1 promoters (FIG. 4). FIG. 4A reflects a schematic diagram of pCEP4d plasmid that replicates in the cell as a multi-copy episome and contains dual H1 promoters to drive expression of the complementary siRNA strands. The plasmid pCEP4 (Invitrogen, Carlsbad, Calif.) was chosen to express the siRNA molecules because of its ability to replicate as a multi-copy episome in mammalian cells. The CMV promoter was removed from the pCEP4 to produce the plasmid pCEP4d, in order to eliminate possible competition between the endogenous CMV promoter and the H1 promoter. Tandem H1 promoters driving expression of sense and antisense siRNA sequences were inserted into pCEP4d as depicted in FIG. 4A. To test transient RNA interference activity by pCEP4d expressing siRNA 6367, the plasmid pCEP4d6367 was transfected into AB12-A2 cells and the level of viral NS3 protein was assessed at 72 h post-transfection by western blot analysis (FIG. 4B). FIG. 4B shows a Western blot evaluation of the levels of HCV nonstructural protein NS3 in AB12-A2 cells. At 72 h post-transfection, AB12-A2 cells containing pCEP4d, pCEP4d6367 or pBS6367 mm were harvested for analysis by western blot. Identical blots were probed with either anti-NS3 (top) or anti-actin (bottom) that served as a control for protein loading.

The level of NS3 was lower in cells containing pCEP4d6367 (FIG. 4B, Lane 2) when compared to cells that were transfected with pCEP4d empty vector (FIG. 4B, Lane 1) or with a plasmid expressing negative control siRNA pCEP4d6367 mm (FIG. 4B, Lane 3). This indicated that the H1 promoter expressed complementary siRNA strands that induced RNA interference directed against HCV. In order to demonstrate stable siRNA activity against HCV in Huh-7 cells, the plasmids pCEP4d6367, pCEP4d6367 mm, or empty vector were introduced into cells by transfection. Cell lines that contained the episomal expression vector were selected using hygromycin for 3 weeks. Following selection and expansion of the hygromycin resistant cell lines, cells were electroporated with 100 ng of HCV subgenomic replicon RNA. Colonies that were resistant to G418, due to the presence of replicon, were counted. HCV-specific RNA interference activity was observed in the cell line containing pCEP4d6367 as evidenced by 70% less G418 resistant colonies (FIG. 4C, Plate 2) compared to control cell lines. Control cells contained the empty vector pCEP4d (FIG. 4C, Plate 1) or the vector expressing siRNA pCEP4d6367 mm (FIG. 4C, Plate 2). FIG. 4C shows stable cell lines that expressed siRNA yielded 75% less HCV replicon-dependent colony growth. Huh-7 cells were transfected with pCEP4d empty vector (top), pCEP4d6367 (middle) or with pCEP4d6367 mm (bottom) and grown for 21 days in media containing hygromycin. After selection, the cells were electroporated with 100 ng of HCVrepAB12neo replicon RNA. Colonies were grown in media containing 800 μg/ml G418 for 14-20 days, fixed, and stained with gentian violet. The numbers of colonies on each plate are shown in parentheses. In each case, the number of colonies represents the average of two independent experiments with the standard deviations.

Thus, potent HCV-specific RNA interference activity could be induced for extended periods of time using cells that constitutively expressed siRNA molecules.

### References

1. Hannon, G. J., Nature, 418:244-251 (2002).
2. Vance, V.; Vaucheret, H., Science, 292:2277-2280 (2001).
3. Cullen, B. R., Nat. Immunol., 3:597-599 (2002).
4. Kim, S. K., Nat. Rev. Genet., 2:681-689 (2001).
5. Clemens, J. C.; Worby, C. A.; Simonson-Leff, N.; Muda, M.; Maehama, T.; Hemmings, B. A.; Dixon, J. E., Proc. Natl. Acad. Sci. USA, 97:6499-6503 (2000).
6. McManus, M. T.; Sharp, P. A., Nat. Rev. Genet., 3:737-747 (2002).
7. Zamore, P. D., Science, 296:1265-1269 (2002).
8. Elbashir, S. M.; Lendeckel, W.; Tuschl, T., Genes. Dev., 15:188-200 (2001).
9. Ui-Tei, K.; Zenno, S.; Miyata, Y.; Saigo, K., FEBS. Lett., 479:79-82 (2000).
10. Elbashir, S. M.; Harborth, J.; Lendeckel, W.; Yalcin, A.; Weber, K.; Tuschl, T., Nature, 411:494-498 (2001).
11. Yu, J. Y.; DeRuiter, S. L.; Turner, D. L., Proc. Natl. Acad. Sci. USA, 99:6047-6052 (2002).
12. Sui, G.; Soohoo, C.; Affar el, B.; Gay, F.; Shi, Y.; Forrester, W. C., Proc. Natl. Acad. Sci. USA, 99:5515-5520 (2002).
13. Miyagishi, M.; Taira, K., Nat. Biotechnol., 20:497-500 (2002).
14. Paul, C. P.; Good, P. D.; Winer, I.; Engelke, D. R., Nat. Biotechnol., 20:505-508 (2002).
15. Paddison, P. J.; Caudy, A. A.; Hannon, G. J., Proc. Natl. Acad. Sci. USA, 99:1443-1448 (2002).
16. Gitlin, L.; Karelsky, S.; Andino, R., Nature, 418:430-434 (2002).
17. Jiang, M.; Milner, J., Oncogene, 21:6041-6048 (2002).
18. Jacque, J. M.; Triques, K.; Stevenson, M., Nature, 418:435-438 (2002).
19. Novina, C. D.; Murray, M. F.; Dykxhoorn, D. M.; Beresford, P. J.; Riess, J.; Lee, S. K.; Collman, R. G.; Lieberman, J.; Shankar, P.; Sharp, P. A., Nat. Med., 8:681-686 (2002).
20. Coburn, G. A.; Cullen, B. R., J. Virol., 76:9225-9231 (2002).
21. Borkhardt, A., Cancer Cell, 2:167 (2002).
22. Kim, W. R., Hepatology, 36:S30-834 (2002).
23. McHutchison, J. G., J. Gastroenterol. Hepatol., 17:431-441 (2002).
24. Lohmann, V.; Korner, F.; Dobierzewska, A.; Bartenschlager, R., J. Virol., 75:1437-1449 (2001).
25. Blight, K. J.; Kolykhalov, A. A.; Rice, C. M., Science, 290:1972-1974 (2000).
26. Krieger, N.; Lohmann, V.; Bartenschlager, R., J. Virol., 75:4614-4624 (2001).
27. Nakabayashi, H.; Taketa, K.; Miyano, K.; Yamane, T.; Sato, J., Cancer Res., 42:3858-3863 (1982).
28. Kolykhalov, A. A.; Agapov, E. V.; Blight, K. J.; Mihalik, K.; Feinstone, S. M.; Rice, C. M., Science, 277:570-574 (1997).
29. Lohmann, V.; Korner, F.; Koch, J.; Herian, U.; Theilmann, L.; Bartenschlager, R., Science, 285:110-113 (1999).
30. Kohler, G.; Milstein, C., Nature, 256:495-497 (1975).
31. Brown, T.; Mackey, K., Current Protocols in Molecular Biology, M., A. F.; Brent, R.; Kingston, R. E.; Moore, D. D.; Seidman, J. G.; Smith, J. A.; Struhl, K. eds., John Wiley & Sons, Inc., 1:491-496 (1997).
32. Lee, N. S.; Dohjima, T.; Bauer, G.; Li, H.; Li, M. J.; Ehsani, A.; Salvaterra, P.; Rossi, J., Nat. Biotechnol., 20:500-505 (2002).
33. Major, M. E.; Rehermann, B.; Feinstone, S. M., Fields Virology, Knipe, D. M.; Howely, P.M. eds., Lippincott, Williams and Wilkins, Philadelphia, 1:1127-1161 (2001).
34. Li, W. X.; Ding, S. W., Curr. Opin. Biotechnol., 12:150-154 (2001).
35. Li, H.; Li, W. X.; Ding, S. W., Science, 296:1319-1321 (2002).
36. Brummelkamp, T. R.; Bernards, R.; Agami, R., Science, 296:550-553 (2002).
37. Barton, G. M.; Medzhitov, R., Proc. Natl. Acad. Sci. USA (2002).
38. Xia, H.; Mao, Q.; Paulson, H. L.; Davidson, B. L., Nat. Biotechnol., 20:1006-1010 (2002).
39. Mercer, D. F.; Schiller, D. E.; Elliott, J. F.; Douglas, D. N.; Hao, C.; Rinfret, A.; Addison, W. R.; Fischer, K. P.; Churchill, T. A.; Lakey, J. R.; Tyrrell, D. L.; Kneteman, N. M., Nat. Med., 7:927-933 (2001).

1. An isolated RNA molecule comprising a first nucleic acid molecule hybrized to a second nucleic acid molecule, the first nucleic acid molecule selected from the group consisting of:
CGU CUA GGC CCC CCG AAC CAC (SEQ ID NO 1)
GCA GAU CCG GGG GGC UUG GUG (SEQ ID NO 2)
CUC GUC CCC UCC GGC CGU ACC (SEQ ID NO 3)
GAG CAG GGG AGG CCG GCA UGG (SEQ ID NO 4)
GGG GGG GAG GCA CCU CAU UUU (SEQ ID NO 5)
CCC CCC CUC CGU GGA GUA AAA (SEQ ID NO 6)
GGA GAU GAA GGC GAA GGC GUC (SEQ ID NO 7)
CCU CUA CUU CCG CUU CCG CAG (SEQ ID NO 8)
GAC ACU GAG ACA CCA AUU GAC (SEQ ID NO 9)
CUG UGA CUC UGU GGU UAA CUG (SEQ ID NO 10)
GGG CAG AAC UGC GGC UAU CGC (SEQ ID NO 11)
CCC GUC UUG ACG CCG AUA GCG (SEQ ID NO 12)

2. An isolated RNA molecule comprising the nucleic acid molecule of SEQ ID NO 1 hybrized to the nucleic acid molecule of SEQ ID NO 2.

3. An isolated RNA molecule comprising the nucleic acid molecule of SEQ ID NO 3 hybrized to the nucleic acid molecule of SEQ ID NO 4.

4. An isolated RNA molecule comprising the nucleic acid molecule of SEQ ID NO 5 hybrized to the nucleic acid molecule of SEQ ID NO 6.

5. An isolated RNA molecule comprising the nucleic acid molecule of SEQ ID NO 7 hybrized to the nucleic acid molecule of SEQ ID NO 8.

6. An isolated RNA molecule comprising the nucleic acid molecule of SEQ ID NO 9 hybrized to the nucleic acid molecule of SEQ ID NO 10.

7. An isolated RNA molecule comprising the nucleic acid molecule of SEQ ID NO 11 hybrized to the nucleic acid molecule of SEQ ID NO 12.

8. The use of a molecule identified in claims 1-7 in the treatment of HCV.

9. A method for reducing the expression of HCV using an isolated RNA molecule according to claims 1-7.

10. A method of treating HCV in a patient comprising administering an effective amount of an isolated RNA molecule according to claims 1-7.

11. A method of inhibiting viral replication of HCV by administering to a patient an effective amount of an isolated RNA molecule according to claims 1-7.

12. A composition comprising an isolated RNA molecule according to claims 1-7 for use in the treatment of HCV.
